(19) United States
(12) Patent Application Publication
(10) Pub. No.: US 2005/0265990 A1
(43) Pub. Date: Dec. 1, 2005

(54) Treatments and nutritional supplements for assisting post-exercise recovery

(76) Inventor: Shawn Talbott, Draper, UT (US)

Correspondence Address: HOLME ROBERTS & OWEN, LLP, 299 SOUTH MAIN, SUITE 1800, SALT LAKE CITY, UT, 84111, US

(21) Appl. No.: 11/140,477

(22) Filed: May 27, 2005

Related U.S. Application Data
(60) Provisional application No. 60/576,048, filed on Jun. 1, 2004.

(51) Int. Cl. A61K 38/46; A61K 31/56; A61K 31/198

(52) U.S. Cl. 424/94.65; 514/171; 514/561; 514/563

(57) ABSTRACT

Compositions are provided for use in post-exercise recovery. An effective amount of leucine, valine and isoleucine in the ratio of 3:1:1 is administered following exercise, and thereafter on a daily basis for some days following exercise.

### CROSS-REFERENCE TO RELATED APPLICATIONS

Priority is claimed to U.S. Provisional Application No. 60/576,048, filed on Jun. 1, 2004, and entitled “Treatments and Nutritional Supplements for Assisting Post-Exercise Recovery,” which application is incorporated herein in its entirety.

### BACKGROUND OF THE INVENTION

1. The Field of the Invention

The present invention is directed to nutritional treatments and supplements for use in assisting in the recovery of humans or animals following exercise.

2. The Relevant Technology

Dietary supplements are routinely used by athletes in their quest for improved athletic performance.

Recently, the concept of enhancing recovery following intense exercise has also received attention within the athletic community. Dietary supplementation with protein bars and carbohydrate beverages has been used effectively to promote immediate post-exercise recovery, but longer-term recovery strategies (days to weeks) are not well developed.

### BRIEF SUMMARY OF THE INVENTION

Compositions are provided for use in post-exercise recovery. An effective amount of leucine, valine and isoleucine in the ratio of 3:1:1 is administered following exercise, and thereafter on a daily basis for some days or weeks following exercise.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

It has been discovered that competitive athletes can obtain substantial benefits from suitable recovery following significant exercise. This allows them to train and compete at a higher level, and also assists to protecting them from the negative effects of overtraining and from illness and injury. The present invention provides targeted dietary supplementation for enhancing recovery following intense exercise.

It has been discovered that consumption of the branched chain amino acids leucine, isoleucine and valine are helpful in post-exercise recovery. Further, it has been discovered that a ratio of approximately 3 parts leucine to approximately 1 part of valine and 1 part of isoleucine is particularly efficacious for post-exercise recovery.

It has also been discovered that a combination of these branched chain amino acids together with glutamine and beta-sitosterol gives surprising results with respect to post-exercise recovery. Addition of papain, bromelain and polyphenols provides yet further benefits.

Surprisingly in view of prior reports regarding the use of various of these materials without the benefit of the teachings herein, it has been discovered that relatively small daily doses of a combination of these substances provides significant benefits. For example, a post-exercise recovery composition including the substances set forth in Table I composition has been found useful for post-exercise recovery.

TABLE I

Leucine (900 mg)
Valine (300 mg)
Isoleucine (300 mg)
Glutamine (1000 mg)
Beta-sitosterol (60 mg)

Another useful composition for post-exercise recovery is set forth in Table II.

TABLE II

Leucine (900 mg)
Valine (300 mg)
Isoleucine (300 mg)
Glutamine (1000 mg)
Beta-sitosterol (60 mg)
Papain (100 mg)
Bromelain (100 mg)
Bioflavonoids extract (30 to 50% polyphenols) (50 mg)

An effective amount of leucine, valine and isoleucine in the ratio of approximately 3:1:1 is advantageously administered shortly following exercise, and thereafter, in order to obtain the benefits of the present invention. Each of the compositions set forth in Tables I and II, respectively, have been found to provide unexpected post-exercise recovery benefits. For significant exercise, such as the running of a marathon, it has been found useful to continue taking daily doses of the supplement for two weeks in order to facilitate post-exercise recovery. Those athletes who engage in frequent exercise can benefit from taking the supplements on an ongoing basis.

The foregoing formulations are illustrative of a post-exercise dosage of the listed materials that has been found to be effective. The listing of precise amounts of the constituents of this composition is not intended to suggest that other formulations would not also provide the desired benefits. On the contrary, one of ordinary skill would be able to make changes in the amounts of these constituents in view of the teachings herein, while maintaining the desired benefits.

Example 1 provides a protocol that has been found to be useful to test compositions in accordance with the present invention, making it possible for those of ordinary skill in the art to test various additional compositions without undue experimentation.

### EXAMPLE 1

A study was conducted to evaluate the effect of a post-exercise recovery supplement containing branched-chain amino acids (BCAAs), glutamine, and beta-sitosterol, on measures of fatigue, mood, cold/flu symptoms, and muscle/joint pain during for the first two weeks following a marathon.

Twenty-five subjects (7 women and 18 men) were randomly assigned to receive the supplement of Table II (S, n=14) or a matching placebo (P, n=11) in a double-blind fashion. Subjects consumed the supplement each dav for 14 days immediately following completion of the Park City Marathon (average elevation 6650 ft). The standardized Profile of Mood States (POMS) questionnaire was used to evaluate Tension, Depression, Anger, Vigor, Fatigue, Confusion and Global Mood State during the 2-week period following the marathon. A standard health log was used to assess daily occurrence and severity of cold/flu symptoms, gastrointestinal complaints and degree of muscle/joint pain. Data were analyzed using two-tailed Student's t-tests with significance set at p<0.05.

S had significantly lower scores for Tension (−31%, p=0.039), Fatigue (−52%, p=0.009) and Confusion (−24%, p=0.020) and higher scores for Vigor (+38%, p=0.005) and Global Mood (+16%, p=0.015) compared to P. There were no differences between groups on measures of Depression or Anger. On measures of cold/flu symptoms, 20% of subjects in P reported symptoms, while none were reported in S. Reports of muscle/joint pain were reduced by 51% in S compared to P (p=0.007) and subjects in S were able to resume normal training an average of 5 days earlier than subjects in P (p=0.033).

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A composition for use in post-exercise recovery comprising effective amounts of leucine, valine and isoleucine in the ratio of 3:1:1, respectively.

2. The composition of claim 1, comprising 900 mg of leucine, 300 mg of valine and 300 mg of isoleucine.

3. A composition for use in post-exercise recovery comprising:
900 mg leucine;
300 mg valine;
300 mg isoleucine;
1000 mg glutamine; and
60 mg of beta-sitosterol.

4. A composition for use in post-exercise recovery comprising:
900 mg leucine;
300 mg valine;
300 mg isoleucine;
1000 mg glutamine;
60 mg of beta-sitosterol;
100 mg papain;
100 mg bromelain; and
50 mg of bioflavonoids extract containing 30 to 50% polyphenols.

5. A method for post-exercise recovery treatment, comprising administering an effective amount of leucine, valine and isoleucine in the ratio of 3:1:1, respectively, following exercise.

6. The method of claim 5, comprising 900 mg leucine, 300 mg valine, and 300 mg isoleucine.

7. The method of claim 5, wherein administration is continued daily following the first administration.

8. A method for post-exercise recovery treatment, comprising administering a composition comprising 900 mg leucine, 300 mg valine, 300 mg isoleucine, 1000 mg glutamine, and 60 mg of beta-sitosterol following exercise.

9. The method of claim 8, wherein administration is continued daily following the first administration.

10. A method for post-exercise recovery treatment, comprising administering a composition comprising 900 mg leucine, 300 mg valine, 300 mg isoleucine, 1000 mg glutamine, 60 mg of beta-sitosterol, 100 mg papain, 100 mg bromelain, and 50 mg of bioflavonoids extract containing 30 to 50% polyphenols following exercise.

11. The method of claim 10, wherein administration is continued daily following the first administration.
